(19) United States
(12) Patent Application Publication
(10) Pub. No.: US 2005/0266027 A1
(43) Pub. Date: Dec. 1, 2005

(54) Live organism product

(76) Inventor: James B. Watson, Pierce, NE (US)

Correspondence Address: THOMTE, MAZOUR & NIEBERGALL, L.L.C., 2120 S. 72ND STREET, SUITE 1111, OMAHA, NE, 68124, US

(21) Appl. No.: 11/055,433

(22) Filed: Feb. 10, 2005

Related U.S. Application Data
(63) Continuation-in-part of application No. 10/853,346, filed on May 25, 2004.

(51) Int. Cl. A61K 39/02; A61K 39/04

(52) U.S. Cl. 424/234.1

(57) ABSTRACT

A live organism product wherein the organisms are in a dormant state and are suspended in a liquid carrier which is substantially moisture-free. The carrier is comprised of oil. The carrier may also include an adsorbent. The product is stored and shipped in a plastic bag and is sprayed onto its target host or the like. The moisture and pH then activates the organisms.

### CROSS-REFERENCE TO RELATED APPLICATION

This is a continuation-in-part application of Petitioner's earlier application Ser. No. 10/853,346 filed May 25, 2004, entitled “LIVE BACTERIA PRODUCT”.

### BACKGROUND OF THE INVENTION 1. Field of the Invention

This invention relates to a dormant organism product and more particularly to a live dormant organism product wherein live organisms, that are in a dormant state, are suspended in a liquid fluid carrier or medium that can be easily pumped and will stay in suspension for the life of the product. More particularly, the carrier, while being in a liquid state, is free of moisture to prevent the organisms from becoming activated until the product is applied to feeds, forages, or directly onto animals or the like. Hereinafter, feeds, forages, animals or the like will be referred to as “the target host”. 2. Description of the Related Art

Live bacteria formulations are frequently applied to forage products, feed, etc., to enhance fermentation and/or digestion. The prior art products fall generally into two categories which are either a dry soluble powder form or a dry granular non-soluble form. The dry granular products are directly applied to its target host through a metering device such as a Gandy box, usually at a rate of 4 to 16 ounces per ton of forage treated. The dry soluble products must first be hydrated with water, then applied to its target host within a 48-hour time period post-hydration with application made through a liquid pump system at the rate of 37 grams to 1 gallon per ton of forage.

The dry granular products of the prior art, even if packaged in plastic pails, will sometimes come into direct contact with ambient air during manufacturing and processing or upon simply opening the product lid with the ambient air containing moisture or humidity. The moisture or humidity will activate the dormant bacteria upon contact which in turn causes the bacteria to inadvertently live and die before it can be applied to the forage. The value of the product will then be a total loss. The limestone carriers of the prior art products will carry minute traces of moisture, and such moisture severely reduces the product's ability to sustain an adequate shelf life. If the prior art product is a soluble powder product, it will also need to be hydrated before application. The hydration immediately activates all of the dormant bacteria and the producer has to apply the entire mixture within a 48-hour time frame or before since the bacteria will naturally deplete its food source and die, creating a total loss of the product value.

Further, the prior art products on the market today must be either removed from their original shipping container or physically poured into a dry applicator prior to application, or they must be contaminated with water, reducing their shelf life to less than 48 hours, post-contamination. Additionally, the prior art dry granular products have a tendency of sorting during shipment. This shakes any fines to the bottom of the shipping container leaving the either larger or lighter material to work its way to the top. Since the bacteria attach themselves to these particles, they may also migrate with these particles. Other dry soluble powder products may settle to the bottom of the applicator tank upon hydration. This will depend greatly on the product's rate of dilution. As application rates decrease to meet the producer's ongoing needs of hauling less product to the field, the manufacturers must in turn make their products more condensed to meet these needs. Such limits the solubility of the product.

The dry granular products of the prior art require a significant percentage of the product to be applied to the material which results in uniform coverage becoming difficult. Dry soluble powders mixed with water carriers have attempted to achieve low inclusion rates for some time. However, such products result in droplets which are very large in their molecular size and weight resulting in that there are actually few droplets being delivered per treatment, an example being one ton of forage treated with only ten large droplets so uniform coverage is poor.

### SUMMARY OF THE INVENTION

A live bacteria product is disclosed wherein the live bacteria are in a dormant state and are suspended in a clear liquid fluid carrier or medium that may easily be pumped and will stay in suspension for the life of the product. More particularly, the carrier or medium for the live bacteria is comprised of a substantially moisture-free liquid which is comprised of: (1) mineral oil and polymers; or (2) a processed oil obtained from either animal, vegetable or petroleum origins. In some cases, the carrier will also have a hydrophilic molecular sieve adsorbent (moisture scavenger) included therein.

The product of this invention is preferably applied to the target host at a micro-treatment rate of 2 to 28 grams per ton of target host being treated. In some cases, more or less percentages may be utilized. The live bacteria, by being suspended in the moisture-free carrier, will remain dormant until coming into contact with the target host at which time the moisture in the target host will cause the bacteria to be activated. The product of this invention is packaged in controlled shipping vessels such as polyurethane bags that reduce the risk of moisture contamination of any kind. The product is sold and shipped directly to the producer who will then take the collapsible bag of product and hook it directly to an applicator which applies the product to the target host. The target host may be forage, feed or the like. In some cases, the product may be sprayed directly onto animals such as chicks who will ingest the product while grooming themselves. Although the product is ideally an improve live bacterial product, the product may be freeze dried live dormant organisms that require ambient moisture to begin proliferation including, but not limited to, modified live bacterial and viral vaccines such as Parvo, Bovine Rhinotracheitis, Leptospira, BVD, IBR and P13 or modified and non-modified pathogens.

It is therefore a principal object of the invention to provide an improved live dormant organism product which requires ambient moisture to begin proliferation.

Yet another object of the invention is to provide a live dormant organism product wherein live organisms, that are in a dormant state, are suspended in a clear liquid fluid carrier or medium that can be easily pumped and will stay in suspension for the life of the product.

Still another object of the invention is to provide a live dormant organism product which is free of moisture to prevent the organism from becoming activated until the product is applied to the target host.

Still another object of the invention is to provide a live dormant organism product which may be applied to a target host at an extremely small rate.

Still another object of the invention is to provide a live dormant organism product wherein the carrier or medium therefore is comprised of a substantially moisture-free liquid comprised of: (1) mineral oil and polymers; or (2) a processed oil obtained from either animal, vegetable or petroleum origins.

Still another object of the invention is to provide a live dormant organism product which will not be activated until coming into contact with moisture associated with the target host.

Yet another object of the invention is to provide a live dormant organism product which may be applied to forage or feed at a preferred micro-treatment rate.

A further object of the invention is to provide a live dormant organism product which is packaged in such a way to limit its exposure to possible contamination from outside sources, such as moisture-laden air or fluid water of any kind.

Still another object of the invention is to provide a live dormant organism product such as modified or non-modified live bacterial and viral vaccines.

These and other objects will be apparent to those skilled in the art.

### DETAILED DESCRIPTION OF THE INVENTION

In the following, the product of this invention is described as a live dormant bacteria product. However, it should be understood that the product could be: (1) a live dormant organism requiring ambient moisture to begin proliferation; (2) modified or live bacterial and viral vaccines such as Parvo, Bovine Rhinotracheitis, Leptospira, BVD, IBR and P13; or (3) modified and non-modified pathogens.

In this invention, conventional live viable harmless bacteria is mixed with a substantially moisture-free liquid carrier or medium. In general, the live bacteria used in the product of this invention will be any lactic acid producing bacteria that is permissible for use in animal related products. The United States Food and Drug Administration (FDA) and The American Association of Feed Control Officials (AAFCO) have published a list of the microorganism species which are “generally recognized as safe” (GRASS) for use in direct-fed microbial products. Table I hereinbelow lists presently approved bacteria for use with animal related products.

TABLE I
36.14--DFM Microorganisms under the “GRASS’ Status

| | |
|---|---|
| Aspergillus niger | Lactobacillus curvatus |
| Aspergillus oryzae | Lactobacillus delbruekii |
| Bacillus coagulans | Lactobacillus fermentum |
| Bacillus lentus | Lactobacillus helveticus |
| Bacillus licheniformis | Lactobacillus lactis |
| Bacillus pumilus | Lactobacillus plantarum |
| Bacillus subtilis | Lactobacillus euterii |
| Bacteroides amylophilus | Leuconostoc mesenteroides |
| Bacteroides capillosus | Pediococcus acidilacticii |
| Bacteroides ruminocola | Pediococcus cervisiae |
| Bacteroides suis | Pediococcus pentosaceus |
| Bifidobacterium adolescentis | Propionibacterium freudenreichii |
| Bifidobacterium animalis | Propionibacterium shermanii |
| Bifidobacterium bifidum | Saccharomyces cerevisiae |
| Bifidobacterium infantis | Entercococcus cremoris |
| Bifidobacterium longum | Entercococcus diacetylactis |
| Bifidobacterium thermophilum | Entercococcus faecium |
| Lactobacillus acidolphilus | Entercococcus intermedius |
| Lactobacillus brevis | |
| Lactobacillus buchneri | Entercococcus lactis |
| Lactobacillus bulgaricus | Entercococcus thermophilius |
| Lactobacillus casei | Yeast (as defined elsewhere) |
| Lactobacillus cellobiosus | |

Preferably, the moisture-free liquid carrier is comprised of a product marketed under the trademark “Synergel®” manufactured by Penreco of 138 Petrolia Street, Karns City, Pa. 16041-9799. The product is generally referred to as a gelled mineral oil, reference number 1004-100, having a Brookfield viscosity of 1242 cPs. The carrier product is clear, odorless and is insoluble in water. The carrier also includes polymers. Other suspension agents such as Cobisal (polymer fiber) may also be used from time to time to aid or reduce the possibility of the settling of the bacteria therein. While the moisture-free oil described above is preferred, a processed oil obtained from either animal, vegetable or petroleum may be used. The product of this invention is designed to only activate the bacteria at the physical point of contact with the target host. Upon contact, the target host ambient moisture content will activate the dormant bacteria found within the liquid microbial medium or carrier. The liquid microbial medium or carrier of this invention has undergone a special production process developed by Penreco. The process consists of first passing the mineral oil through an advanced filtration system which specifically targets and absorbs any ambient moisture. Secondly, the mineral oil is heated and polymers are added to increase the oil viscosity and suspension qualities. Thirdly, the oil is packaged into controlled shipping vessels that reduce the risk of moisture contamination of any kind.

Upon receipt of the packaged carrier, applicant then preferably adds a moisture scavenger product such as a hydrophilic molecular sieve adsorbent as an insurance program. The adsorbent may comprise a natural or synthetic zeolite consisting of crystalline metal aluminosilicate, alkali metal aluminosilicate or sodium aluminosilicate. Finally, whatever bacteria are needed for a particular purpose will then be added. The final blend is then packaged in moisture and ultraviolet retardant containers such as collapsible polyurethane bags, very similar to I.V. bags. The product is then sold and shipped directly to the retailer and/or end user. The end user will then take the collapsible bag of liquid microbial blend and hook it directly to an applicator so that extremely small droplets are created which are sprayed upon the target host

The invention described herein provides the ability to inoculate other products with dormant live bacteria, by means of a light spraying application. Whereas the bacteria is protected with the oil/polymer blend allowing the host bacteria to survive longer in a non-favorable environment, the coating of the bacteria cell walls with the oil/polymer covering provides a physical chemical moisture barrier. The above provides the ability to permit the live bacteria to be introduced onto a pelleted feed without immediately inadvertently activating the oil/polymer/host bacteria through ambient moisture contamination from the feed itself.

The product of this invention permits it to be applied to its target host at a preferred micro-treatment rate of 2 to 28 grams per one ton of forage or the material treated (with no additional water or carriers needed). In some cases, higher or lower ratios may be employed. Such micro-treatment is substantially less than prior art products that have treatment rates that range from 37.88 grams to 8.3453 pounds per one ton of forage treated.

The product of this invention and its packaging limits its exposure to possible contamination from outside sources, such as moisture-laden air or fluid water of any kind. The product of this invention is designed to only activate at the physical point of contact with the target host. Upon contact with the target host, the change in pH and moisture activates the dormant bacteria.

Thus it can be seen that the invention accomplishes at least all of its stated objectives.

1. The method of forming stabilized admixtures of dormant viable organisms, comprising:
(a) preparing an admixture by intermixing a substantially moisture-free liquid carrier and the dormant organism.

2. The method of claim 1 wherein said substantially moisture-free liquid carrier is selected from either mineral oil and polymers or a processed oil obtained from animal, vegetable or petroleum origins.

3. The method of claim 1 wherein said admixture includes a hydrophilic molecular sieve adsorbent.

4. The method of claim 3 wherein said adsorbent comprises a natural or synthetic zeolite.

5. The method of claim 4 wherein said zeolite comprises crystalline metal aluminosilicate.

6. The method of claim 4 wherein said zeolite comprises alkali metal aluminosilicate.

7. The method of claim 4 wherein said zeolite comprises sodium aluminosilicate.

8. The method of claim 1 wherein the admixture is sprayed upon a target host with the moisture and pH causing the activation of the organism.

9. The method of claim 8 wherein the admixture is sprayed upon the target host at a rate of 2 to 28 grams per ton of forage material.

10. The method of claim 1 wherein the admixture is sprayed upon a target host with the ambient moisture and pH causing the activation of the organism.

11. The method of claim 8 wherein the admixture is sprayed upon the target host at a rate of 2 to 28 grams per ton of feed material.

12. A stabilized admixture for application to feed, forage, or animals or the like, comprising:
a substantially moisture-free liquid carrier; and
dormant viable organisms suspended in said carrier.

13. The admixture of claim 12 wherein said substantially moisture-free liquid carrier is comprised of oil.

14. The admixture of claim 12 wherein the admixture includes a hydrophilic molecular sieve adsorbent.

15. The admixture of claim 14 wherein said adsorbent comprises a natural or synthetic zeolite.

16. The admixture of claim 15 wherein said zeolite comprises crystalline metal aluminosilicate.

17. The admixture of claim 15 wherein said zeolite comprises alkali metal aluminosilicate.

18. The admixture of claim 15 wherein said zeolite comprises sodium aluminosilicate.

19. The admixture of claim 12 wherein the admixture is sprayed upon the target host with the moisture and pH in the target host causing the activation of the organism.

20. The admixture of claim 19 wherein the admixture is sprayed upon the target host at a rate of 2 to 28 grams per ton of forage material.

21. The admixture of claim 12 wherein the admixture is sprayed upon a target host with the moisture and pH in the target host causing the activation of the organism.

22. The admixture of claim 21 wherein the admixture is sprayed upon the target host at a rate of 2 to 28 grams per ton of target host.

23. The admixture of claim 12 wherein the admixture is sprayed onto the target host.

24. The method of claim 1 wherein the admixture is sprayed onto a target host.

25. The admixture of claim 12 wherein the organism comprises a live bacterial vaccine.

26. The admixture of claim 12 wherein the organism comprises a live viral vaccine.

27. The admixture of claim 12 wherein the organism comprises a modified or non-modified pathogen.
